(19) United States
(12) Patent Application Publication
(10) Pub. No.: US 2022/0013229 A1
(43) Pub. Date: Jan. 13, 2022

(54) REMOTE TESTING AND FITTING SYSTEM FOR HEARING

(71) Applicant: Nautilus Consulting, LLC, Altoona, IA (US)

(72) Inventors: Kevin Jasa, Urbandale, IA (US); Steven Bennethum, West Des Moines, IA (US)

(21) Appl. No.: 17/373,068

(22) Filed: Jul. 12, 2021

Related U.S. Application Data
(60) Provisional application No. 63/050,367, filed on Jul. 10, 2020.

(51) Int. Cl. G16H 40/67; H04R 25/00; A61B 5/12; A61B 1/227; G16H 80/00; G16H 10/20; G16H 40/20; G09B 5/06

(52) U.S. Cl. CPC G16H 40/67; H04R 25/70; A61B 5/123; A61B 1/227; H04R 2225/55; G16H 10/20; G16H 40/20; G09B 5/065; G16H 80/00

(57) ABSTRACT

A remote testing and fitting system for hearing and method of using the same that includes a kit having hearing aids, foam tip inserts, a video scope and a bone oscillator. The hearing aids, video scope and bone oscillator are all connected to a software application that is downloaded to an electronic device. The software application is adapted to conduct both hearing tests and fitting procedures for the hearing aids.

### CROSS REFERENCE TO RELATED APPLICATION

This application claims the benefit of U.S. Provisional Application No. 63/050,367 filed Jul. 10, 2020, the contents of this application is hereby incorporated by reference in its entirety.

### BACKGROUND OF THE INVENTION

The present invention is directed to a testing and fitting system for hearing, and more particularly a system for hearing where testing and fitting are performed either automatically, or by a hearing practitioner, remotely.

Typically, to test a person's hearing, the person schedules an appointment with a hearing practitioner where questions are answered and tests are performed to determine if the person is a candidate for hearing aids. If they are, a hearing aid fitting is conducted where the hearing aids are digitally adjusted to preferred settings. This process can be inconvenient, particularly for elderly patients, where transportation to practitioners can be difficult, and, in light of COVID-19, unsafe. The ability to conduct testing a fitting remotely in real time is therefore needed.

Accordingly, an objective of the present invention is to provide a system capable of conducting remote hearing tests and fitting in real time.

Another objective of the present invention is to provide a system that is capable of conducting remote hearing tests and fittings automatically.

These and other objectives, features, and advantages of the invention will become apparent from the specification and claims.

### BRIEF DESCRIPTION OF THE DRAWINGS

FIG. 1 is a schematic view of an environment of a testing and fitting system for hearing; and

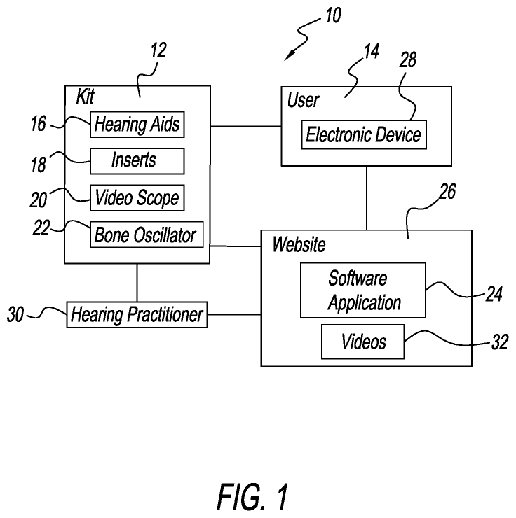

FIG. 2 is a flow diagram of a testing and fitting system for hearing.

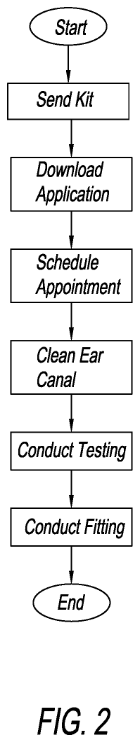

### SUMMARY OF THE INVENTION

A remote testing and fitting system for hearing and method of using the same includes a kit having hearing aids, foam tip inserts, a video scope, and a bone oscillator. The hearing aids, video scope and bone oscillator are all connected to a software application that is downloaded to an electronic device. The software application is also adapted and/or configured to conduct both a hearing test and a fitting procedure for the hearing aids. The software and the hearing aids are calibrated and adapted to conduct accurate pure tone, speech reception threshold, most comfortable level and uncomfortable level testing.

The software application permits the user to input adjustments to the hearing aids or is programed to operate automatically without the use of a hearing practitioner. As an example the system is programed to prompt the user to input feedback and then the software application makes digital adjustments to the hearing aids based upon the user feedback.

### DETAILED DESCRIPTION

Referring to the Figures, a remote testing and fitting system for hearing 10 includes a kit 12 that is sent to a user or patient 14. The kit 12 includes, but is not limited to, hearing aids 16, foam tip insert receivers 18, a video scope 20, a bone oscillator 22 and fitting and cleaning accessories. The kit 12 also includes access to a downloadable software application 24, preferably downloaded from an app store 26 to an electronic device 28 such as a smartphone, personal computer, or tablet. The hearing aids 16 are preferably calibrated and adapted to conduct accurate pure tone, speech reception threshold (SRT), most comfortable level (MCL) and uncomfortable level (UCL) testing. The foam tip inserts 18 are used to close off the ear canal of the user to reduce background noise. The hearing aids 16, video scope 20, and bone oscillator 22 all are connected to the downloadable software application 24 via Bluetooth or wired. The downloadable software 24 also is adapted to conduct a fitting procedure for the hearing aids 16.

Upon receipt of the kit 12, the user 14 downloads the downloadable software application 24 from the app store 26 to an electronic device 28. Initially, the user 14 is prompted by the software application 24 to answer a series of questions, sign applicable health related waivers, and schedule a remote appointment with a hearing practitioner 30. For the remote appointment the hearing practitioner 30 and the user 14 are connected through the software application 24 by video and/or audio. Initially, the user 14 is instructed by the practitioner 30 to check for wax in the ear canal using the video scope 20. The video scope 20 transmits video to the practitioner 30 who determines if the ear canal is clear of wax. If not, the practitioner 30 provides instructions on where to go to remove wax from the ear canal.

Next, the user 14 inserts the hearing aids 16 with the foam tip insert receivers 18 into the ear canal and using the software application 24 the pure tone, MCL, UCL, and SRT testing is conducted. As a result, from a remote location, the practitioner 30 is able to receive and analyze results in real time. In addition, the practitioner 30 is also able to remotely conduct a fitting procedure whereby through the software 24 digital adjustments are made to the hearing aids 16. The practitioner 30 can also walk the user 14 through a bone testing using the bone oscillator where the bone oscillator transmits information through the software application 24 to the practitioner 30.

In addition to permitting remote real time testing and fitting, the software application 24 includes a plurality of instructional videos 32. The videos 32 address information concerning how to use hearing aids, trouble shooting for common problems, how to clean hearing aid videos, a testing how to video and how to make adjustments. The software application 24 also permits user programing where volume can be turned up and down, adjustment of a three to six channel equalizer, change programs, adjust for wind noise reduction, background noise reduction, microphone directionality, frequently asked questions and options to help fix issues, use of some of the basic portions of the testing and fitting programing, surveys on how the adjustment helps patients, and the ability to save old settings.

In another embodiment, the downloadable software application 24 is programed to operate automatically without the use of a hearing practitioner 30. In this embodiment, the software application 24 prompts the user 14 to perform the same steps outlined above. For example, the software application 24 would prompt the user 14 to examine the ear canal for wax using the video scope 22 and based upon information received from the video scope 24 the software application would provide feedback on whether the ear canal was clear and if not would provide instructions on where to get the ear canal cleaned. With respect to testing and fitting, the user would be prompted to input feedback based upon the testing and the software application would provide feedback and/or make digital adjustments to the hearing aids based upon the user feedback.

From the above discussion and accompanying figures and claims it will be appreciated that the remote testing and fitting system for hearing 10 offers many advantages over the prior art. It will be appreciated further by those skilled in the art that other various modifications could be made to the device without parting from the spirit and scope of this invention. All such modifications and changes fall within the scope of the claims and are intended to be covered thereby. It should be understood that the examples and embodiments described herein are for illustrative purposes only and that various modifications or changes in the light thereof will be suggested to persons skilled in the art and are to be included in the spirit and purview of this application.

What is claimed is:

1. A remote testing and fitting system for hearing, comprising:
a kit that includes hearing aids, foam tip inserts, a video scope and a bone oscillator;
an electronic device configured to download a software application wherein the hearing aids, video scope and bone oscillator are connected to the software application; and
wherein the software is adapted to conduct a hearing test and a fitting procedure for the hearing aids.

2. The system of claim 1 wherein the hearing aids are calibrated and adapted to conduct accurate pure tone, speech reception threshold, most comfortable level, and uncomfortable level testing.

3. The system of claim 1 wherein the software application includes a plurality of instructional videos.

4. The system of claim 3 wherein the plurality of instructional videos provide information concerning how to use hearing aids, trouble shooting for common problems, how to clean hearing aid, how to test hearing aids, and how to make adjustments.

5. The system of claim 1 wherein the software application permits a user to input adjustments to the hearing aids.

6. The system of claim 1 wherein the software application is programed to operate automatically without the use of a hearing practitioner.

7. The system of claim 6 wherein the software application prompts a user to input feedback and the software application then would make digital adjustments to the hearing aids based upon the user feedback.

8. A method of remotely testing and fitting for hearing, comprising the steps of:
providing a kit that includes hearing aids, foam tip inserts, a video scope and a bone oscillator;
downloading a software application to an electronic device;
connecting the user and the hearing practitioner for the remote appointment through the software application;
determining if the user's ear canal is clear of wax using the video scope which transmits video to the hearing practitioner through the software application;
inserting the hearing aids with the foam tip inserts into the ear canal of the user and using the software application testing a plurality of tones; and
transmitting the test results using the software application in real time.

9. The method of claim 8 further comprising the step of providing instructions on removing wax from the ear canal when it is determined that a user's ear canal is not clear of wax.

10. The method of claim 8 wherein the plurality of tones include accurate pure tone, speech reception threshold, most comfortable level, and uncomfortable level.

11. The method of claim 8 further comprising the step of conducting a fitting procedure where through the software application adjustments are made to the hearing aids.

12. The method of claim 8 further comprising the step of conducting bone testing using the bone oscillator and transmitting the bone oscillator information to the hearing practitioner through the software application.

13. The method of claim 8 further comprising the step of prompting a user to answer a series of questions, sign applicable health related waivers, and schedule a remote appointment with a hearing practitioner with the software application.
